SEQUENCE LISTING (21 sequences)

SEQ ID NO: 1 - DNA, 20 bp, Homo sapiens
agtccctcttcactttcctc

SEQ ID NO: 2 - DNA, 20 bp, Homo sapiens
gcttttcccttcttcttttc

SEQ ID NO: 3 - DNA, 20 bp, Homo sapiens
attttcatacctttgctgga

SEQ ID NO: 4 - DNA, 20 bp, Homo sapiens
taacagcttcaacacctcct

SEQ ID NO: 5 - DNA, 20 bp, Homo sapiens
gagctggatgagaacaacac

SEQ ID NO: 6 - DNA, 38 bp, Homo sapiens
actgaacctgaccgtacaaaagaagtggcaggaagagt

SEQ ID NO: 7 - DNA, 49 bp, Homo sapiens
ctgcaggagttctttgaccacatacagttccctgatgagtccgtgagga

SEQ ID NO: 8 - DNA, 45 bp, Homo sapiens
actagttggtgcagttatatgcagcagatttcgtcctcacggact

SEQ ID NO: 9 - DNA, 49 bp, Homo sapiens
ctgcagacaagcaacaccactgcacttctttctgatgagtccgtgagga

SEQ ID NO: 10 - DNA, 52 bp, Homo sapiens
actagttatatgcagcagatcggaactgtatgtggtttcgtcctcacggact

SEQ ID NO: 11 - DNA, 44 bp, Homo sapiens
ctgcaggtgcagctgtgggagctcttctgatgagtccgtgagga

SEQ ID NO: 12 - DNA, 56 bp, Homo sapiens
actagtgctaggggaagaggcgctcctcccccaccaccttttcgtcctcacggact

SEQ ID NO: 13 - protein, 505 aa, Homo sapiens
MSSVQQQPPPPRRVTNVGSLLLTPQENESLFTFLGKKCVTMSSAVVQLYAADRNCMWSKKCSGVACLVKDNPQRSHFLRIFDIKDGKLLWEQELYNNFVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKGDIGTPSNFQHIGHVGWDPNTGSDLNNLDPELKNLFDMCGILEAQLKERETLKVIYDFIEKTGGVEAVKNELRRQAPPPPPPSRGGPPPPPPPPHSSGPPPPPARGRGAPPPPPSRAPTAAPPPPPPSRPSVEVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWED

SEQ ID NO: 14 - DNA, 1792 bp, Homo sapiens
ggcagagggacaacgaccatccggccctagcctggccgggcgggtgccgggagcttccctttctcagcgcggcggaaggtggctcgccgtcagcgcctgcttccctcgacctcgtcctcctccccgctccggacgagccgagatgtggcgcctctgactccacttctccccgcccctgtcaccgagagggggaacgagctctcgcccactcgccggagagacggccctggactcccaaccccgccggcgaaaccatgagctccgtccagcagcagccgccgccgccgcggagggtcaccaacgtggggtccctgttgctcaccccgcaggagaacgagtccctcttcactttcctcggcaagaaatgtgtgactatgtcttcagcagtggtgcagttatatgcagcagatcggaactgtatgtggtcaaagaagtgcagtggtgttgcttgtcttgttaaggacaatccacagagatctcattttttaagaatatttgacattaaggatgggaaactattgtgggaacaagagctatacaataactttgtatataatagtcctagaggatattttcatacctttgctggagatacttgtcaagttgctcttaattttgccaatgaagaagaagcaaaaaaatttcgaaaagcagttacagaccttttgggccgtcgacaaaggaaatctgagaaaagacgagatcccccaaatggtcctaatctacccatggctacagttgatataaaaaatccagaaatcacaacaaatagattttatggtccacaagtcaacaacatctcccataccaaagaaaagaagaagggaaaagctaaaaagaagagattaaccaagggagatataggaacaccaagcaatttccagcacattggacatgttggttgggatccaaatacaggctctgatctgaataatttggatccagaattgaagaatctttttgatatgtgtggaatcttagaggcacaacttaaagaaagagaaacattaaaagttatatatgactttattgaaaaaacaggaggtgttgaagctgttaaaaatgaactgcggaggcaagcaccaccacctccaccaccatcaaggggagggccacctcctcctcctccccctccacatagctcgggtcctcctcctcctcctgctaggggaagaggcgctcctcccccaccaccttcaagagctcccacagctgcacctccaccaccgcctccttccaggccaagtgtagaagtccctccaccaccgccaaataggatgtaccctcctccacctccagcccttccctcctcagcaccttcagggcctccaccaccacctccatctgtgttgggggtagggccagtggcaccacccccaccgcctccacctccacctcctcctgggccaccgcccccgcctggcctgccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagactgatctatatattatatatatac

SEQ ID NO: 15 - protein, 502 aa, Homo sapiens
MSGGPMGGRPGGRGAPAVQQNIPSTLLQDHENQRLFEMLGRKCLTLATAVVQLYLALPPGAEHWTKEHCGAVCFVKDNPQKSYFIRLYGLQAGRLLWEQELYSQLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 16 - DNA, 1796 bp, Homo sapiens
gagaagacaagggcagaaagcaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgagtggctgagttacttgctgccctgtgctcctccccgcaggacatggctccccctccacctgctctgtgcccaccctccactctcctcttccaggcccccaaccccccatttcttccccaccaacccctccaatgctgttatccctgcctggtcctcacactcacccaacaatcccaaggccctttttatacaaaaattctcagttctcttcactcaaggatttttaaagaaaaataaaagaattgtctttctgtctctctata

SEQ ID NO: 17 - DNA, 83 bp, Homo sapiens
ctgcaggagttctttgaccacatacagttccctgatgagtccgtgaggacgaaatctgctgcatataactgcaccacactagt

SEQ ID NO: 18 - DNA, 89 bp, Homo sapiens
ctgcagacaagcaacaccactgcacttctttctgatgagtccgtgaggacgaaaccacatacagttccgatctgctgcatataactagt

SEQ ID NO: 19 - DNA, 87 bp, Homo sapiens
ctgcaggtgcagctgtgggagctcttctgatgagtccgtgaggacgaaaaggtggtgggggaggagcgcctcttcccctagcctagt

SEQ ID NO: 20 - DNA, 18 bp, Homo sapiens
atgagctccgtccagcag

SEQ ID NO: 21 - DNA, 21 bp, Homo sapiens
tcagtcttcccactcatcatc

(19) United States
(12) Patent Application Publication
(10) Pub. No.: US 2016/0038563 A1
(43) Pub. Date: Feb. 11, 2016

(54) MOLECULAR TARGETS FOR HEALING OR TREATING WOUNDS

(71) Applicant: University College Cardiff Consultants Limited, Cardiff (GB)

(72) Inventors: Keith Harding, South Glamorgan (GB); Wen Guo Jiang, South Glamorgan (GB)

(21) Appl. No.: 14/752,087

(22) Filed: Jun. 26, 2015

Related U.S. Application Data
(62) Division of application No. 13/579,999, filed on Aug. 20, 2012, now abandoned.

(30) Foreign Application Priority Data
Mar. 5, 2010 (GB) 1003652.3

(51) Int. Cl. A61K 38/12; A61K 9/00; C12N 15/113; A61K 31/403

(52) U.S. Cl. CPC A61K 38/12; A61K 31/403; A61K 9/0014; C12N 15/113; A61K 48/00

(57) ABSTRACT

The invention relates to at least one molecular target for healing or treating wounds and, in particular chronic, human wounds. The molecular target is nWASP or a protein at least 75% homologous therewith and which retains the same activity as nWASP protein, such as WASP. Further, the invention concerns a novel therapeutic for treating said wounds and a novel gene therapy approach, involving said molecular target, for treating said wounds.

This application is a divisional patent application of U.S. patent application Ser. No. 13/579,999 filed on Aug. 20, 2012, which in turn is the national stage of international patent application no. PCT/GB2011/050298 filed on Feb. 16, 2011 and claiming priority to British Patent Application Serial No. GB1003652.3 filed on Mar. 5, 2010, the disclosures of each of which are incorporated herein by reference in their entirety.

### TECHNICAL FIELD

The present invention relates to at least one molecular target for healing or treating wounds and, in particular, human wounds. More particularly still, the molecular target has application in the treatment of chronic wounds. Further, the invention concerns a novel therapeutic for treating said wounds and a novel gene therapy approach, involving said molecular target, for treating said wounds. Additionally, the invention concerns a method for treating wounds using said therapeutic or said gene therapy.

### BACKGROUND

In one form or another, chronic and poorly healing wounds constitute a major burden on the UK health system. Moreover, in certain member countries of the EU health expenses relating to wound healing are already approaching the third most expensive drain on health care funding.

Chronic foot ulcers are a major complication of diabetes, accounting for up to 25% of all hospital admissions involving diabetes, and at a cost to the UK National Health Service of £250M annually. Chronic foot ulcers cause substantial morbidity, impair the quality of life, and are the major cause of lower limb amputation. Despite careful attention to foot care, as many as 25% of diabetics develop foot ulcers in their lifetimes. The causes of lower limb ulceration are the same in diabetics as in non-diabetics, namely neuropathy, ischaemia and trauma. However, this “pathogenic triad” predisposes wounds to infection, which can also contribute to the non-healing nature of the wounds.

Current treatment involves removing pressure from the area, debridement, wound dressing and management of infection: surgical resection and vascular reconstruction may be required in more advanced disease, which ultimately may necessitate amputation.

In addition to lower limb ulcers in diabetics, another major resource health cost is created by pressure wounds or ulcers that result, for example, from failure to provide routine nursing or medical care. In the UK 412,000 people are affected annually by this sort of wound at a cost of £1.4-2.1 billion.

The healing of a wound is controlled by complex biological processes that involve a diverse number of cell types; complex interactions between cells and tissues; the activation of the immune system and the activation of the angiogenic process. Moreover, all of these processes involve a large number of molecules.

A typical healing process can be divided into 5 distinct, but closely related, stages: clotting stage, acute inflammation stage, matrix deposition stage, capillary formation stage and re-epithelialisation stage. A diverse number of factors are involved in and control each of these stages. Deficiencies in any aspect of the process may result in defective wound healing. Thus, a ‘normal’ healing process may be defective as a result of either intrinsic or external factors, which manifest as ‘abnormal non-healing’ or ‘chronic’ wounds. It is these chronic or ‘non-healing’ wounds that present the greatest challenge to the quality of a patient's life and mounting expenses to the healthcare system.

Although some common clinical/pathological factors may assist in pre-judging if a wound may be ‘healing’ or ‘non-healing’, or if an acute wound may become chronic, there is no specific laboratory test(s) to distinguish wound type. Additionally, there is no clear way to define how to predict the healing process and a patient's likely response to treatment in chronic wound care.

### WASP/n-WASP

Wiskott-Aldrich syndrome (WAS), also described as Werlhof's disease (Van den Bosch and Drukker 1964), was originally described in American kindred where it was manifested as eczema, thrombocytopenia, proneness to infection, and bloody diarrhoea (Alfrich et al. 1954). Death usually occurs before the age of 10 years. The causes of death are mainly infections or bleeding, but also development of malignancies: lymphoreticular tumors and leukemia reticuloendothelial system malignancies (Perry et al. 19980, Ten Bense et al 1966, Sullivan et al. 1994).

It has been recognised that the Wiskott-Aldrich protein provides a link between Cell Division Cycle 42 (Cdc42) and the actin cytoskeleton (Symons et al. 1996). T lymphocytes of affected males with WAS exhibit a severe disturbance of the actin cytoskeleton, suggesting that the WAS protein may regulate its organization. The WAS protein interacts with Cdc42, a member of the RHO family of GTPases (thus GTP-dependent) it was detected in cell lysates, in transient transfections, and with purified recombinant proteins (Kolluri et al. 1996) suggesting that the WAS protein functions as a signal transduction adaptor downstream of Cdc42, and that cytoskeletal abnormalities may result from a defect in Cdc42 signalling. It has since been demonstrated that WAS is a rare X-linked disorder with variable clinical phenotypes that correlate with the type of mutations in the WAS protein (WASP) gene (Ochs and Thrasher 2006).

It has also been shown that WASP is a key regulator of actin polymerization in hematopoietic cells with 5 domains involved in signalling, cell motility/migration, in immune synapse formation and in facilitating the nuclear translocation of nuclear factor kappaB (Ochs and Thrasher 2006). Mutations of WASP are located throughout the gene and either inhibit or dysregulate normal WASP function: classic WAS occurs when WASP is absent; X-linked thrombocytopenia when mutated WASP is expressed; and X-linked neutropenia when missense mutations occur in the Cdc42-binding site (Ochs and Thrasher 2006).

Miki et al (1996) first described a 65 kDa protein from brain that bound to the SH3 domains of Ash/Grb2. The amino acid sequence was approximately 50% homologous to Wiskott-Aldrich syndrome protein (WASP) and was termed N-WASP (neural-WASP). N-WASP has several functional motifs (such as a pleckstrin homology (PH) domain and cofilin-homologous region) through which N-WASP depolymerizes actin filaments. N-WASP-stimulated actin assembly is responsible for membrane ruffling (Zalevsky et al., 2001), a process that actively involves the cytoskeletal associated protein family, ERM (ezrin-moesin-radixin)(Brescher et al 1989, Hiscox and Jiang 1999). N-WASP activity is regulated by an intramolecular interaction that is alleviated following concomitant binding of Cdc42-GTP to the Cdc42/Rac interactive binding (CRIB) domain and PtdIns(4,5)P₂ to the polybasic region (Kovacs et al. 2006). We have recently reported that two major complexes that linked to the WASP family, namely the ERM family and Rho GTPases were aberrantly expressed in human breast cancer (Harrison et al 2003, Jiang et al 2003).

Thus the two homologous proteins share a common functionality in stimulating actin assembly or organisation of the cytoskeleton.

In our investigations we have surprisingly discovered what may be termed a loss in the control of nWASP activities in chronic, or abnormal, wounds where nWASP is over-expressed compared with acute wounds which have low levels of nWASP expression. This observation indicated to us the potential role of nWASP or nWASP-like proteins in the healing process. We therefore undertook work to block the aberrantly expressed nWASP in chronic wounds and found it helped in the healing of keratinocytes. Further work showed that manipulating nWASP is an effective and safe way to promote the healing of difficult wounds where nWASP is over-expressed and this over-expression results in a hindered healing process.

In summary, we have identified at least one molecular target for treating wounds and in particular human wounds. More particularly, but not exclusively, said molecular target has application in the treatment of chronic wounds. The first molecular target is nWASP and therefore the invention relates to a novel therapeutic comprising an inhibitor of either, or both, nWASP expression or nWASP activity. In the former instance, the invention involves a novel gene therapy approach and in the latter instance a novel protein therapy approach. Further, given the sequence homology and structure and function homology between n-WASP and WASP the molecular target of the invention further extends to WASP. Accordingly, the invention also relates to a novel therapeutic comprising an inhibitor of either, or both, WASP expression or WASP activity. In the former instance, the invention involves a novel gene therapy approach and in the latter instance a novel protein therapy approach.

Reference herein to nWASP, or WASP, is reference to a gene or protein whose identity is shown in FIG. 13.

Our invention can improve the quality of a patient's life by ensuring that new wounds do not deteriorate into a chronic state and existing chronic wounds can be treated in a way that actively promotes healing.

### SUMMARY

Accordingly, in one aspect of the invention there is provided a novel therapeutic comprising an inhibitor of either, or both, nWASP gene expression or nWASP protein activity.

In the former instance, the invention involves a novel gene therapy approach and in the latter instance a novel protein therapy approach. Thus, in one embodiment the novel therapeutic comprises an inhibitor of nWASP gene expression, this inhibitor can be anti-sense DNA, anti-sense RNA, siRNA, or ribozymes, either naked or in the form of plasmid and viral vectors. Those skilled in the art are aware of the aforementioned inhibitory molecules and so would be able to work the invention once they knew that over-expression of nWASP contributed to the chronic wound phenotype. However, in another embodiment the novel therapeutic comprises an inhibitor of nWASP protein function, this inhibitor can be either a nWASP binding agent that binds, either reversibly or irreversibly, to inhibit protein function such as an antibody or a known, or synthesized, nWASP antagonist; or an agent that works upstream or downstream of the nWASP signalling mechanism to inhibit nWASP signalling and so negate the effects of over-expression of nWASP protein in chronic wound tissue. Those skilled in the art are aware of the aforementioned inhibitory molecules and so would be able to work the invention once they knew that over-expression of nWASP contributed to the chronic wound phenotype.

Additionally, or alternatively, the invention also comprises a novel therapeutic comprising an inhibitor of either, or both, WASP gene expression or WASP protein activity.

As above, in the former instance, the invention involves a novel gene therapy approach and in the latter instance a novel protein therapy approach. Thus, in one embodiment the novel therapeutic comprises an inhibitor of WASP gene expression, this inhibitor can be either anti-sense DNA or RNA, siRNA, or ribozymes, either naked or in the form of plasmid and viral vectors. Those skilled in the art are aware of these inhibitory molecules and so would be able to work the invention once they knew that over-expression of WASP contributed to the chronic wound phenotype. However, in another embodiment the novel therapeutic comprises an inhibitor of WASP protein function, this inhibitor can be either a WASP binding agent that binds, either reversibly or irreversibly, to inhibit protein function such as an antibody or a known, or synthesized, WASP antagonist; or an agent that works upstream or downstream of the WASP signalling mechanism to inhibit WASP signalling and so negate the effects of over-expression of WASP protein in chronic wound tissue. Those skilled in the art are aware of the aforementioned inhibitory molecules and so would be able to work the invention once they knew that over-expression of WASP contributed to the chronic wound phenotype.

In an embodiment of the invention the therapeutic comprises an nWASP gene inhibitor such as transgene 1 or transgene 2 or transgene 3 described herein. These molecules are termed anti-nWASP ribozyme/RNA transgenes. Transgene 1 is produced by transcription of the nWASP gene using the following short oligos:

(SEQ ID NO 7)
nWASPRib1F (5′Ctgcaggagttctttgaccacatacagttccctgat
gagtccgtgagga′3)
and
(SEQ ID NO 8)
nWASPRib1R (5′ActagttggtgcagttatatgcagcagAtttcgtcc
tcacggact′3).

Transgene 2 is produced by transcription of the nWASP gene using the following short oligos:

(SEQ ID NO 9)
nWASPrib2F (5′Ctgcagacaagcaacaccactgcacttctttctgat
gagtccgtgagga′3)
and
(SEQ ID NO 10)
nWASPRib2R (5′ActagttatatgcagcagATCggaactgtatgtgGt
ttcgtcctcacggact′3).

Transgene 3 is produced by transcription of the nWASP gene using the following short oligos:

(SEQ ID NO 11)
nWASPRib3F (5′Ctgcaggtgcagctgtgggagctcttctgatgagtc
cgtgagga′3)
and
(SEQ ID NO 12)
nWASPRib3R (5′Actagtgctaggggaagaggcgctcctcccccacca
ccTtttcgtcctcacggact′3).

These products are antisense-hammerhead ribozyme also known as antisense-hammerhead RNA, ideally they are flanked by selected restriction sites such as pstI and SpeI and more ideally still they are cloned into a cloning vector such as pEF6/V5/His TOPO.

The sequence structure of transgene 1 is:

(SEQ ID NO 17)
5′Ctgcaggagttctttgaccacatacagttccctgatgagtccgtgagg
acgaaatctgctgcatataactgcaccacactagt′3.

The sequence structure of transgene 2 is:

(SEQ ID NO 18)
5′Ctgcagacaagcaacaccactgcacttctttctgatgagtccgtgagg
acgaaaccacatacagttccgatctgctgcatataactagt′3.

The sequence structure of transgene 3 is:

(SEQ ID NO 19)
5′Ctgcaggtgcagctgtgggagctcttctgatgagtccgtgaggacgaa
aaggtggtgggggaggagcgcctcttcccctagcctagt′3.

In an embodiment of the invention the therapeutic comprises a commercially available nWASP protein inhibitor such as, without limitation, Wiskostatin (Merck Pharmaceuticals) or 187-1 (TOCRIS).

In a further embodiment of the invention the therapeutic comprises a commercially available WASP protein inhibitor.

The therapeutic of the invention is for use in treating mammalian wounds, ideally chronic, and, more ideally still, human.

An antibody for use in the invention is most ideally a monoclonal antibody or a humanised antibody.

In the above aspects and embodiments of the invention the therapeutic is formulated for topical application.

Alternatively, in the above aspects and embodiments of the invention the therapeutic is formulated for oral application.

Alternatively again, in the above aspects and embodiments of the invention the therapeutic is formulated for application to a dressing or impregnation in a dressing.

The therapeutic of the invention may be administered in combination with an antibiotic or antibacterial agent. Numerous such agents are known and suitable choices will be familiar to skilled practitioners.

In yet another aspect of the invention, there is provided a pharmaceutical composition comprising a therapeutic of the invention together with a pharmaceutically acceptable carrier.

Other active materials may also be present in the pharmaceutical composition, as may be considered appropriate or advisable for the wound being treated. For example, the composition may also contain an emollient, or the like.

The carrier, or, if more than one be present, each of the carriers, must be acceptable in the sense of being compatible with the other ingredients of the formulation and not deleterious to the recipient.

The formulations include those suitable for topical (including eye drops), oral (including buccal and sublingual), rectal, nasal or vaginal administration and may be prepared by any methods well known in the art of pharmacy.

The composition may be prepared by bringing into association the therapeutic of the invention and the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a therapeutic of the invention in conjunction or association with a pharmaceutically or veterinarily acceptable carrier or vehicle.

For topical application to the skin, compounds of conventional use may be made up into a cream, ointment, jelly, solution or suspension etc. Cream or ointment formulations that may be used for the composition are conventional formulations well known in the art, for example, as described in standard text books of pharmaceutics such as the British Pharmacopoeia.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

Other formulations suitable for oral administration include lozenges comprising the active agent in a flavoured or inert base base and mouthwashes comprising the active agent in a suitable liquid carrier.

In a further aspect of the invention there is provided a method for treating a mammalian wound, typically a chronic wound, which method comprises:

administering to said wound a therapeutic that inhibits either, or both of, nWASP gene expression or nWASP protein activity.

Additionally, or alternatively, the further aspect of the invention also, or alternatively, comprises a novel method for treating a mammalian wound, typically a chronic wound, which method comprises:

administering to said wound a therapeutic that inhibits either, or both of, WASP gene expression or WASP protein activity.

According to yet a further aspect of the invention there is provided a kit for treating a wound, preferably a chronic wound, wherein said kit comprises:

(a) at least one therapeutic as above described; and
(b) at least one dressing for applying to said wound.

According to a yet further aspect of the invention there is provided a combination therapeutic comprising an inhibitor of n-WASP gene expression and an inhibitor of WASP gene expression.

According to a yet further aspect of the invention there is provided a combination therapeutic comprising an inhibitor of n-WASP protein activity and an inhibitor of WASP protein activity.

According to a yet further aspect of the invention there is provided a combination therapeutic comprising an inhibitor of: a) either n-WASP or WASP gene expression; and an inhibitor of b) either n-WASP or WASP protein activity.

According to a further aspect of the invention there is provided a therapeutic comprising an inhibitor of nWASP, or a homologoue thereof.

According to a further aspect of the invention there is provided use of an inhibitor of nWASP, or a homologoue thereof, in the manufacture of a medicament for treating a wound.

According to a further aspect of the invention there is provided use of an inhibitor of nWASP, or a homologue thereof, for treating a wound.

The term “homologue” as used herein refers to amino acid sequences which have a sequence at least 50% homologous to the amino acid sequence of nWASP and which retain the biological activity of the nWASP sequence. In embodiments, homologues are at least 75% homologous to the nWASP peptide sequence and, in increasing order of preference, at least 80%, 85%, 90%, 95% or 99% homologous to the nWASP peptide sequence.

Treatment of a wound described herein includes reference to human or veterinary use.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

Features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

### BRIEF DESCRIPTION OF THE DRAWINGS

The present invention will now be described by way of the following examples with particular reference to FIGS. 1-21 wherein:

FIG. 1. Shows expression analysis of nWASP transcript in normal skin, acute wound and abnormal/chronic wound tissues. Shown are median and the inter-quartile range of the transcript level;

FIG. 2. Shows Left: The predicted secondary structure of human nWASP which was the basis for designing the anti-nWASP rizozyme transgenes. Right: The effect of anti-nWASP transgenes on levels of nWASP mRNA after transfection. Transgenes-1 2 and 3 were active in knocking down the nWASP transcript from HaCaT cells;

FIG. 3. Shows frequency scanning of HaCat cells. Probe-A2: medium control; probe-B3: Wiskostatin 100 nM; probe-B6: Wiskostatin 10 nM; probe-B7: Wiskostatin 1 nM;

FIG. 4. Shows effects of Wiskostatin on the adhesiveness of HaCaT cells in an electric wounding assay. Probe-A2: medium control; probe-A3: Wiskostatin 100 nM; probe-A6: Wiskostatin 10 nM; probe-A7: Wiskostatin 1 nM. Shown are 400 (top), 4000 (middle) and 40,000 Hz (bottom);

FIG. 5. Shows effects of Wiskostatin on HGF-induced adhesion of HaCaT cells in an electric wounding assay. All wells included HGF at 40 ng/ml except where it is indicated. Probe-B2: medium control; probe-B3: Wiskostatin 100 nM; probe-B6: Wiskostatin 10 nM; probe-B7: Wiskostatin 1 nM. Shown are 400 (top), 4000 (middle) and 40,000 Hz (bottom). Probe-A2: no HGF control;

FIG. 6. Shows effects of Wiskostatin on the migration of HaCaT cells in an electric wounding assay. Probe-A2: medium control; probe-A3: Wiskostatin 100 nM; probe-A6: Wiskostatin 10 nM; probe-A7: Wiskostatin 1 nM. Shown are 400 (A-top), 4000 (B-middle) and 40,000 Hz (C-bottom);

FIG. 7. Shows effects of Wiskostatin on HGF-induced the migration of HaCaT cells in an electric wounding assay. Probe-B2: medium control; probe-B3: Wiskostatin 100 uM; probe-B6: Wiskostatin 10 uM; probe-B7: Wiskostatin 1 uM. Shown are 400 H. All wells except indicated were with HGF 40 ng/ml;

FIG. 8. Shows effects of an nWASP inhibitor, 187-1, on the migration and micromotion of HaCaT cells. Wild type HaCaT cells were treated with either control medium (A2), 187-1 at 10 μM (A3), 187-1 at 1.0 μM (A6), or 187-1 at 0.1 μM (A7). The migration was recorded using ECIS9600 over a period up to 3 hours. The inhibitor has substantially increased the speed of migration/healing with the most obvious effect seen at 10 μM. (graph generated from Experiment ID 20 Feb. 2009 HaCaT 187-1). A-shows traces of cell migration; B-shows the migration as calculated by Rb cell modelling (* p=0.0114 vs control); C: Concentration dependent stimulation of migration by 187-1; D-shows the modelling of micromotion, 8 p<0.05 vs control;

FIG. 9. Shows evaluation of the role of nWASP in the migration of HaCaT keratinocytes. Top Left: A1 is the wild type HaCaT; A2 is HaCaT cells transfected with anti-nWASP transgene-2 and A3 with ant-nWASP transgene-3. A4 is cell free electrode control. Loss of nWASP from the cells (A2 and A3; also refer to FIG. 2) resulted in dramatic increase in cell migration (A2 and A3 vs A1). Top Right: all wells included HGF at 40 ng/ml. A5 is the wild type HaCaT; A6 is HaCaT cells transfected with anti-nWASP transgene-2 and A7 with ant-nWASP transgene-3. A8 is cell free electrode control; Bottom two graphs: the effect of nWASP inhibitors on the growth of HaCaT cells (bottom left-Wiskostatin, Bottom right-187-1): Wiskostatin had limited inhibitory effect on the growth and only at a high concentration. 187-1 had no significant effect on the growth of the cells;

FIG. 10. Shows nWASP had a profound impact on the function of endothelial cells. A: Cellular migration after nWASP expression. Electrode-A1: HECV wild type cells; A2: HECV/pcDNA-GFP plasmid control cells; A3/A4: duplicated assay for HECV transfected with pcDNA/GFP-nWASP7 plasmid. Forced expression of nWASP markedly reduced the migration of the cells. B: Quantified changes as shown by resistance in OHM. There was a significant reduction in the migration speed as shown by reduced resistance in nWASP over-expressing cells (p<0.05 vs wild type and control cells);

FIG. 11. Shows A: Tubule formation 24 hours (X4 magnification). A1-A4-HECV-wt, B1-B4-HDCV-GFP control, C1-C4: HECV/nWASP7exp cells. 1-control; 2-cell treated with 187-1 (10 uM, except C2A=1 uM, and C2B-10 uM); 3-cells treated with Wiskostatin shown are 100 nM; 4-positive control (cells treated with HGF at 50 ng/ml). Arrows indicate microtubules. Shown are micrographs 24 hours after seeding. B: The same experimental setting but photographed after 48 hours at ×10 magnification;

FIG. 12 Shows the effects of blocking nWASP by nWASP inhibitor 187-1 on the expansion of chronic wound tissue (WD). 187-1 treated in the expansion in areas of the chronic tissues compared with the control tissues during the 24 hour period;

FIG. 13 shows the amino acid and cDNA sequence structure of nWASP and WASP;

FIG. 14 shows nWASP expression was significantly higher in wound tissues that fail to heal (chronic non-healing, n=51) than in wound that heal within 3 months (chronic healed, n=20);

FIG. 15 shows the effect of systemic administration of 187-1 on the change of the size of wounds, shown are the percentage change of wound areas over day-1 in the respective groups;

FIG. 16 shows the effect of systemic administration of 187-1 on the size of wounds, shown are size of the wounds in pixels (mean±SD);

FIG. 17A shows topical application of 187-1 by Gel-B reduced the size of wounds, shown is the percentage change of wound areas over day-1 in the respective groups;

FIG. 17B shows topical application of 187-1 by Gel-B reduced the size of wounds, shown are size of the wounds in pixels (mean±SD);

FIG. 18A shows topical delivery of 187-1 by Gel A, shown is the percentage change of wound areas over day-1 in the respective groups;

FIG. 18B shows topical application of 187-1 by Gel-A reduced the size of wounds, shown are size of the wounds in pixels (mean±SD);

FIG. 19A shows the effect of systemic administration of Wiskostatin on wound healing, at both concentrations, there was a significant reduction of the size of wound by Wiskostatin, shown is the percentage change of wound areas over day-1 in the respective groups;

FIG. 19B shows the effect of systemic administration of Wiskostatin on wound healing, at both concentrations, there was a significant reduction of the size of wound by Wiskostatin, shown are size of the wounds in pixels (mean±SD);

FIG. 20A shows topical application of Wiskostatin on wound healing, shown are the percentage change of wound areas over day-1 in the respective groups;

FIG. 20B shows topical application of Wiskostatin on wound healing, shown are the percentage change of wound areas over day-1 in the respective groups, shown are size of the wounds in pixels (mean±SD);

FIG. 21A shows topical application of Wiskostatin using Gel A, shown is the percentage change of wound areas over day-1 in the respective groups; and

FIG. 21B shows topical application of Wiskostatin using Gel A, shown is the percentage change of wound areas over day-1 in the respective groups.

### DETAILED DESCRIPTION

### Materials and Procedure

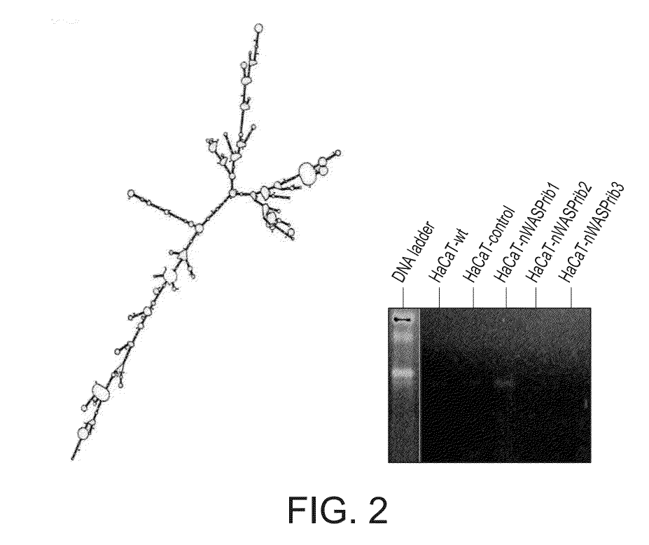

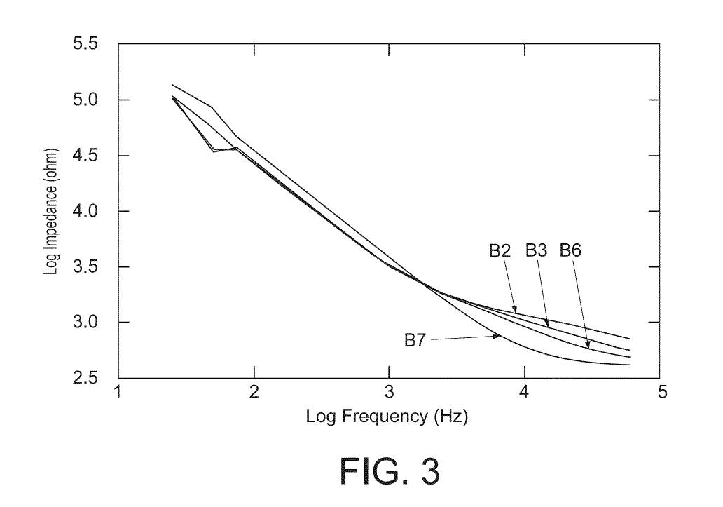

1. Cells and Human keratinocytes and melanoma Cells (HaCaT-from The German Cancer Institute/Cell Service, Germany and A431—from ATCC), human vascular endothelial cell, HECV (from Interlab, Italy) were used. Recombinant human HGF was from the research laboratory. nWASP inhibitors, Wiskostatin and 187-1 were from Merck Pharmaceuticals and TOCRIS, respectively. Tissue collection and preparation of RNA/cDNA bank from human wound/skin tissues. Fresh tissues from abnormal/chronic wounds (n=14), acute wounds (n=10) and normal skin from healthy volunteers (n=10) were collected under an approval from the local ethical committee (Ethical approval ID: 05/WSE03/92) and stored in −80° C. until use. Written informed consent was obtained from each patient who agreed for a biopsy to be taken. Tissues were frozen sectioned on a cryostat (Leica). A portion of the sections were kept for histological analysis. Approximate 20 sections were pooled and homogenised using a hand-held homogenizer using a procedure to extract RNA from the tissues. RNA extracted from the tissues was quantified and cDNA was subsequently generated using a RT kit. Abnormal/chronic wound tissues were from patients with abnormal/chronic leg ulcers. Acute wound tissues were obtained from patients with acute surgical wounds after undergoing excision of pilonidal disease. Normal tissues were from normal volunteer's normal skin.
2. Analysis of nWASP gene transcripts was carried out using conventional and quantitative real time PCR (Icycler IQ, Bio-Rad) on human wound and skin tissues and cells. For conventional PCR, primers used were: NWASPF8 (5′agtccctatcactttcctc′3) (SEQ ID NO 1) and NWASPR8 (5′gcttttcccttcttcttttc′3) (SEQ ID NO 2) and NWASPF9 (5′attttcatacctttgctgga′3) (SEQ ID NO 3) and NWASPR9 (5′taacagcttcaacacctcct′3) (SEQ ID NO 4). For Q-PCR primers used were nWASPF1 and NWASPZR1 (5′gagctggatgagaacaacac′3) (SEQ ID NO 5) and (5′actgaacctgaccgtacaaaagaagtggcaggaagagt′3) (SEQ ID NO 6).
3. The study adopted the Ampliflor quantitation technology, in which one set of gene specific primers and a Uniprimer probes were used, in combination with quantitative PCR master mix. The reaction was carried out using ICycler^{IQ} (Bio-Rad). An internal standard was employed for quantitation purpose. In all the assays, GAPDH and actin were amplified and used as the house keeping controls. Levels of a specific gene was normalised to the expression level of GAPDH in the respective sample for example, nWASP, GAPDH and actin transcripts (mRNA) were measure in the same sample. nWASP was then normalised to GAPDH.
4. Evaluation of cells response to wound stress after n-WASP is targeted. Here, an in vitro cell model, based on the ECIS system, was adopted. Briefly, cells were allowed to sit in the electric arrays and an 8E10 format was used. Cells in fixed number and fixed volume of culture medium were added into each of the respective wells, which had either control medium or a specific inhibitor to N-WASP, namely Wiskostatin or 187-1. In selected wells, a cell migration inducer, HGF was also included in the absence or presence of the nWASP inhibitors. Cells were investigated for the following functions: the adhesiveness to the matrix surface and the migration capacity. The first was recorded immediately after plating of the cells into each well at 3 frequencies: 400 Hz, 4,000 Hz, and 40,000 Hz. The frequencies were chosen following a frequency scanning (FIG. 3). Two main cell functions were thus tested here: the adhesiveness of the cells to matrix and cell migration.
5. Creation of cell models. We have created a new cell model, based on a human keratinocyte cell line, HaCaT, which expressed high levels of n-WASP. By using anti-nWASP transgenes, we successfully knocked out the expression of n-WASP from the cells and subsequently used the cells in our testing. Briefly, the secondary structure (FIG. 2) of human nWASP was generated using Zuker's RNA mFold software. Three suitable sites for targeting within the nWASP mRNA were identified. These sites fulfil the following criteria: GTC/ATC/TTC sequence in the mRNA sequence and GTC/ATC sequence situated in a loop (a large loop preferred) region and not in a stem region. Touch-down PCR was used to generate PCR-based ribozyme/RNA using paired oligos. PstI and SpeI restriction sites were introduced during the PCR reaction. Three anti-nWASP transgenes were prepared using the following short oligos: nWASPRib1F (5′Ctgcaggagttctttgaccacatacagttccctgatgagtccgtgagga′3) and nWASPRib1R (5′ActagttggtgcagttatatgcagcagAtttcgtcctcacggact′3), nWASPrib2F (5′ Ctgcagacaagcaacaccactgcacttattctgatgagtccgtgagga′3) and nWASPRib2R

(5′ActagttatatgcagcagATCggaactgtatgtgGtttcgtcctcac
ggact′3),

(5′Ctgcaggtgcagctgtgggagctcttctgatgagtccgtgagga′3) and nWASPRib3R (5′ActagtgctaggggaagaggcgctcctcccccaccaccTtttcgtcctcacggact′3).

PCR products generated antisense-hammerhead ribozyme, flanked by pstI and SpeI restriction sites, were T-A cloned into a pEF6/V5/His TOPO cloning vector which used EF6 promoter for mammalian expression (Invitrogen, Paisley, Scotland, UK), which was amplified in the ONESHOT™ E. coli (Invitrogen). Clones with correct oriented insert were verified using PCR. Plasmid was subsequently purified from the bacterial preparation and used for transfection of HaCaT cells by way of electroporation (EasyJet, Flowgen, England, UK). Following selection, transgenes-1, 2 and 3 were found to be active in knocking down nWASP mRNA from HaCaT cells.
6. For cell adhesion assay, we adopted the ECIS methods by employing ECIS9600 and 1600R models of these instruments. Cells were plated into the corresponding wells in a 8W10E array, in which different concentration of Wiskostatin was included (please see Figure legends). The adhesion was recorded at 400 Hz, 4,000 Hz and 40,000 Hz over a 3 hour period.
7. Cell migration assay. This was essentially the same as the adhesion assay, except that cells were allowed to reach confluence first. They were then electrically wounded at 5 volts for 60 seconds. Electrical sensing was immediately applied after wounding for over a period up to 15 hours, at varying frequencies. Furthermore, we also determined the micromotion of cells under the same experimental setting. This is one further type of function that our instrument measures, in that quiescent cells (no wounding) were evaluated by the instrument over a period of minutes at a 0.1 second interval. The instrument would predict the micromotion between cell membrane and the electrodes. Micromotion reflects the subtle interaction between cell membrane and electrode and partly reflects the migration capacity of the cells. Micromotion was recorded at 15 minute intervals and analysed using cell modelling.
8. Ex vivo effects of nWASP inhibitors in chronic human wound tissues. This was based on an ex vivo model that we previously established (Jiang and Harding 1998). Briefly, fresh biopsies from abnormal/chronic wounds were immediately placed in a purposely made buffer that mimic the physiological fluid and with a mixture of antibiotics. The tissues were finely minced using sterile scalpel to sizes below 1 mm in diameter. After extensive washing in the buffer, the living tissues were immediately embedded in extracellular matrix gel as we previously described in Jiang W G and Harding K G. Enhancement of expansion of wound tissue and angiogenesis by matrix embedded fibroblasts (Dermagraft), a role for hepatocyte growth factor/scatter factor. International Journal of Molecular Medicine, 1998, 2 (2), 203-210. The gels and the topping solution include test materials (cytokines and nWASP inhibitors). The tissues were photographed daily. The degree of tissue expanded from the implanted tissues was calculated using the imager as we previously reported in Jiang W G and Harding K G. Enhancement of expansion of wound tissue and angiogenesis by matrix embedded fibroblasts (Dermagraft), a role for hepatocyte growth factor/scatter factor. International Journal of Molecular Medicine, 1998, 2 (2), 203-210.
9. nWASP and angiogenesis. We used an in vitro angiogenesis model, endothelial tubule forming assay. Briefly, endothelial cells with differential expression of nWASP were sandwiched between layers of matrix proteins and allowed to form microtubules. The cells were also treated with known angiogenic factors or nWASP inhibitors. The tubules were evaluated using a time lapse video recorder and image analysis tools. The endothelial cells used in this study, HECV, which were negative in nWASP expression, were transfected with a mammalian expression plasmid that carried full length human nWASP coding region. This was prepared from normal breast tissue cDNA ((using primer sets: 5′atgagctccgtccagcag′3, SEQ ID NO 20; and 5′tcagtcttcccactcatcatc′3 (SEQ ID NO 21)) was T-A cloned into an pcDNA-NT GFP-TOPO (Invitrogen) plasmid, selection marker G418). The impact of nWASP expression and nWASP inhibitors on tubule formation was evaluated. Furthermore, cell functions including cell attachment and cell migration after forced expression of nWASP in HECV cells were also determined using the ECIS method.
10. Statistical analysis was conducted using Minitab, SPSS and an online Chi-square service tool (www.people.ku.edu/˜preacher/chisq/chisq.htm).

### In Vivo Studies

### In Vivo Tolerance Test.

First, the main tolerance tests were conducted using the CD-1 athymic mice (Charles River Laboratories), owing to their slow and steady rate of growth and easier to observe changes in the skin (hairless) and other possible side effects. Briefly, CD-1 of 4-6 weeks old, 20 g in weight, were housed in filter topped cages. 187-1 (MW 1784, dissolved in BSS buffer) and Wiskostatin (MW 426, dissolved in DMSO and diluted in BSS), were injected, via the intraperitoneal route, on a daily basis. Both compounds were given at 1 and 10M final concentration in 100 ul in volume. Dosages administered were 1 and 10 uM for each compounds, equivalent to 1.8 g/kg/day and 17.8 g/kg/day for 187-1 and 0.43 g/kg/day and 4.3 g/kg/day. CD-1 were observed daily, weighed twice weekly. An additional tolerance and efficacy test was carried out using the db/db strain.

### In Vivo Efficacy Test and Wound Healing.

The diabetic strain of db/db was obtained from Harlan. 4-6 weeks old with body weight at 20 g were used. Creation of a wound was according to a recently described method (Cho et al 2006). Briefly, after being housed for a week, the db/db mice were first ear-pieced using an ear puncher, in order to create a wound (hole) of 1 mm in diameter. The following day after wound creation, all the db/db were weighed, wound photographed using a digital camera. Treatment was given systemically (by IP injection) or topically (by manually applying the compounds in gel into the wound area). Both treatments were given every other day. Images were obtained weekly. The size of the wounds was determined using an image analysis software. Data are given in two ways:

1. the area of the wounds in pixels. Two sample student t test was used for statistical analysis.
2. change of the size of wound over the starting point calculated using: (area at a given point—area at the starting point)/(area of the starting point)×100. Bonferroni model was used for data analysis.

### Formulation of the Compounds.

For systemic application, 187-1 was dissolved in BSS and diluted in the same for the required concentration. Wiskostatin was first dissolved in DMSO at concentration of 5 mg/ml. The DMSO solution was then gradually diluted in BSS in order to avoid precipitation. The solutions were prepared such that each 100 ul contained the correct amount of compounds and was aliquatted and stored at −20° C. until used. The compounds were injected every other day by the IP route. Dosages administered were 0.5 and 5 uM or 0.89 g/kg/day and 8.9 g/kg/day for 187-1 and 1 and 10 uM or 0.43 g/kg/day and 4.3 g/kg/day. Treatment was given every other working day (Monday, Wednesday and Friday) and the images obtained on Wednesday.

For topical application, we used two conventional carrier gels (purchased from Pharmacy of University Hospital of Wales) that are currently used in wound care. From the concentrated master stock of 187-1 and Wiskostatin, 100 ul of the stock solution was mixed with 2 grams of the respective gels (equivalent to 1 mg of the respective compound for 1 gram of the respective gel), followed by low speed homogenisation using a hand held homogeniser, for 2 minutes. The newly formulated gels which showed no sign of changes of the strength and consistency, were stored at 4° C. until used. For use, a small amount (150 ul) of the gel was applied to the wound area and gently rubbed in using fingers. Treatment was given every other working day (Monday, Wednesday and Friday) and the images obtained on Wednesday.

### Results

### N-WASP is Differentially Expressed in Normal Skin, Acute and Abnormal/Chronic Wound Tissues

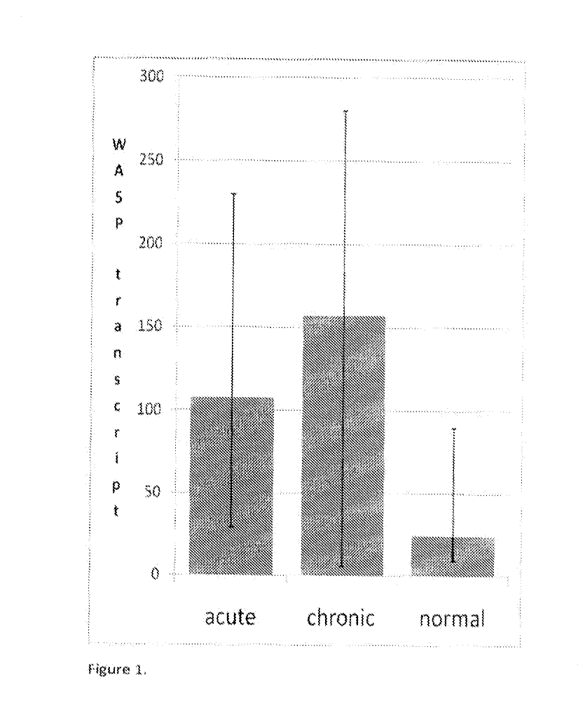

By examining the levels of nWASP mRNA transcripts in human skin and wound tissues, it was revealed that abnormal/chronic wound tissues had significantly higher levels of the nWASP transcripts than normal skin and acute tissues (FIG. 1).

### N-WASP Inhibitor, Wiskostatin Increase the Capability of Cell Adhesion to Matrix

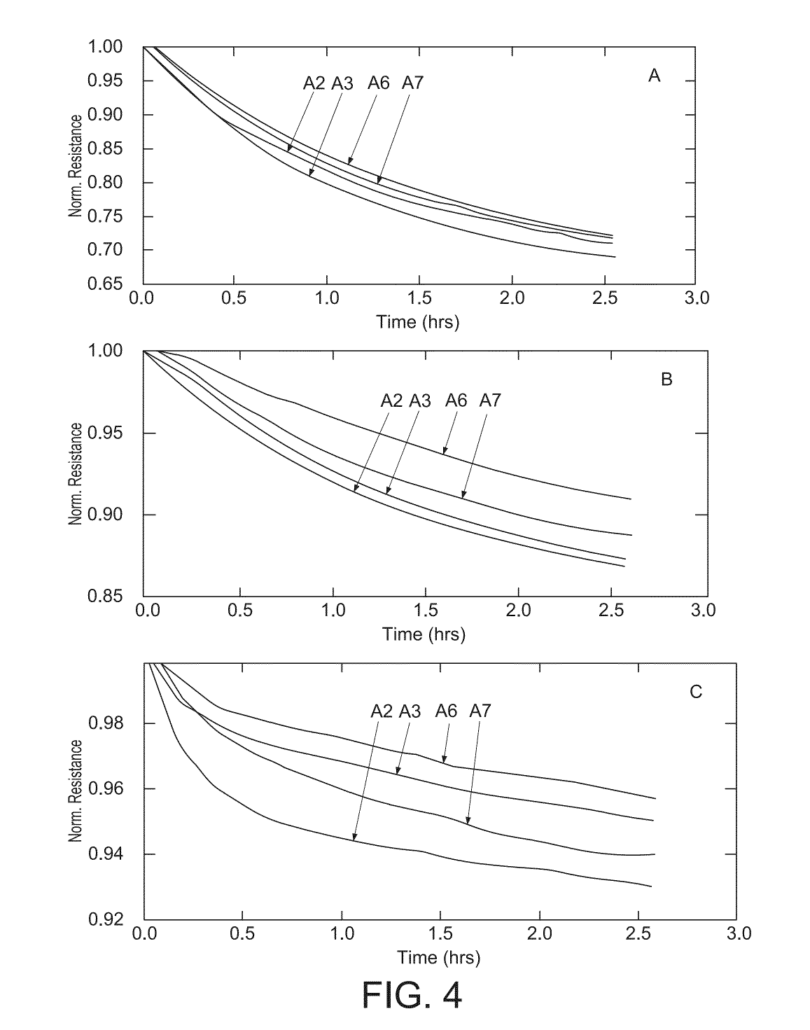

HaCaT cell expressed nWASP as shown by conventional RT-PCR (FIG. 2-Right). In FIG. 2 right, there is a faint band indicating the presence of nWASP transcript in wild type, control and in transgene-3 cells, indicating that transgene-3 was not very active. As shown in FIG. 4, inclusion of Wiskostatin increased the adhesiveness of HaCaT cells to matrix surface, particularly when recorded at 40,000 Hz, at which minor differences between cells are visible (please see FIG. 3).

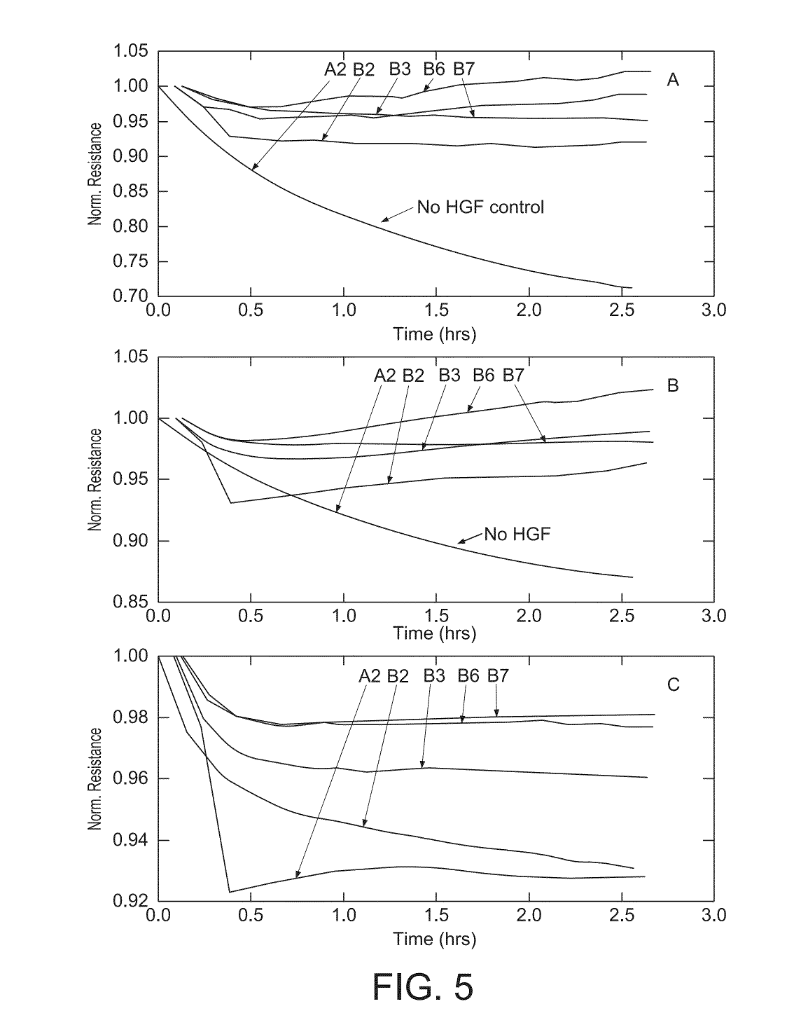

As shown in FIG. 5, where cells were treated with HGF, a cytokine that increases the motility of cell, HGF increased the adhesiveness of the cells compared with control cells. Inclusion of Wiskostatin further increased the adhesiveness, as shown by a rapid return to normal level after the cells were wounded.

### Wiskostatin Increased the Migration of HaCat Cells

Using a similar assay, we further evaluated the impact of inhibition of N-WASP on the migration of HaCaT cells.

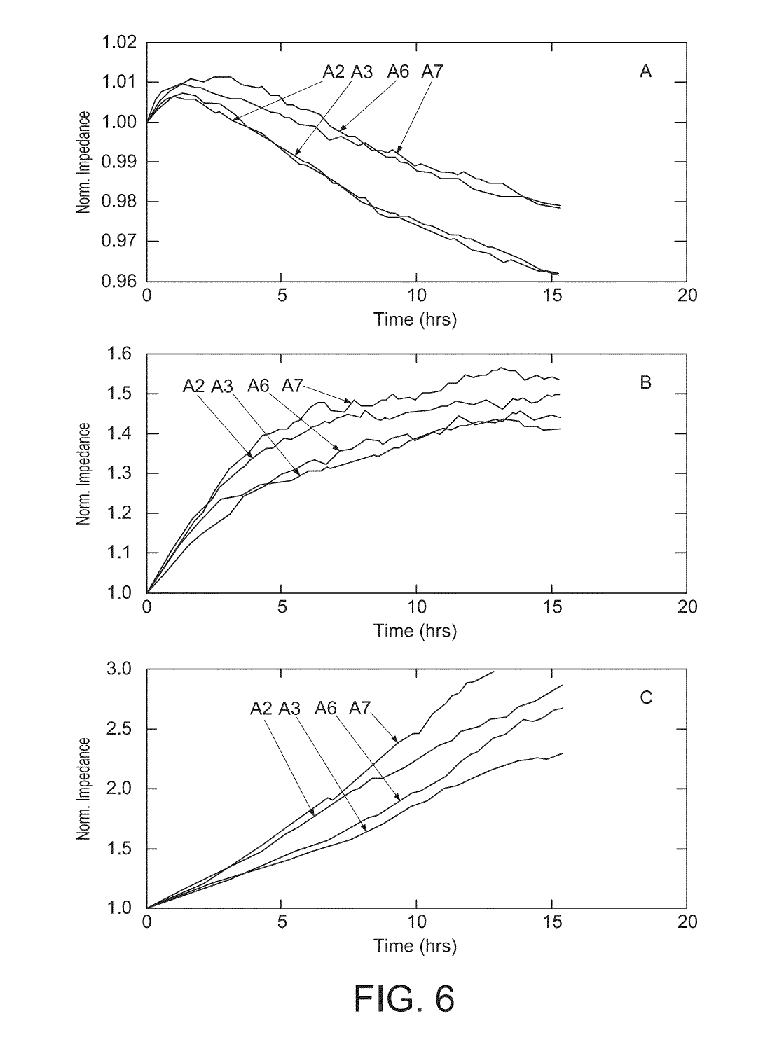

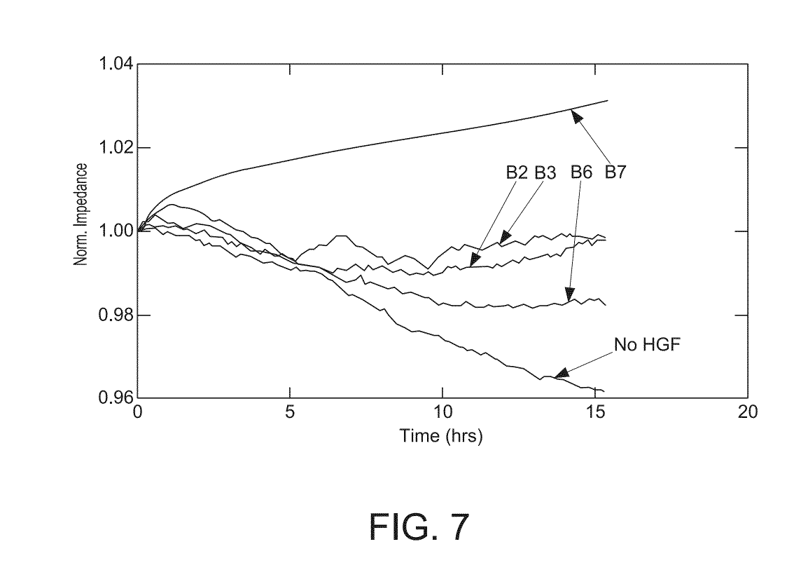

FIGS. 6 and 7 shows the effects of Wiskostatin on the migration of HaCat cells. For example, measured at 400 Hz (FIG. 6A), inclusion of Wiskostatin at 1 nM and 10 nM resulted in a rapid rise of resistance, following wounding.

187-1, an nWASP Inhibitor Displayed a Similar Stimulatory Effect on the Healing of the Keratinocyte Monolayer

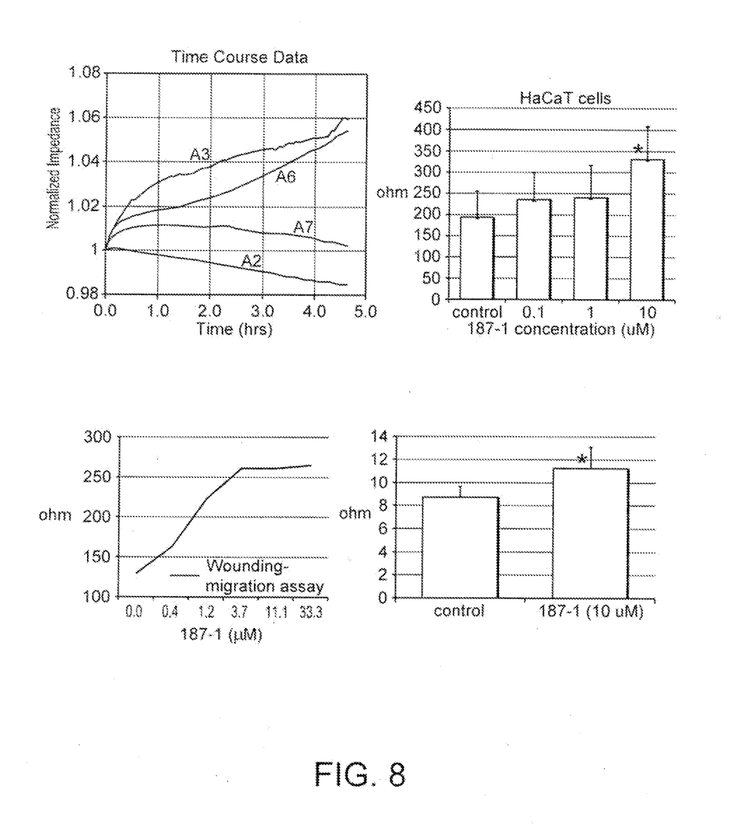

Using the above methods, we tested the effect of 187-1, a small nWASP inhibitor (TOCRIS, MW 1784), on the migration of HaCaT cells. As shown in FIG. 8a, over a concentration range tested, 187-1 showed a stimulatory effect on the migration of the cells with the strongest effect seen at 10 μM. The statistical difference was demonstrated using the Rb Cell Modelling methods in which cells with 10 μM 187-1 showed a rapid increase in migration speed (p=0.0114) (FIG. 8B). Furthermore, a concentration dependent effect was seen with the effect reaching maximum after 10 μM (FIG. 8C). Micromotion analysis has revealed that cells given 187-1 also had a significant increase in micromotion (FIG. 8D).

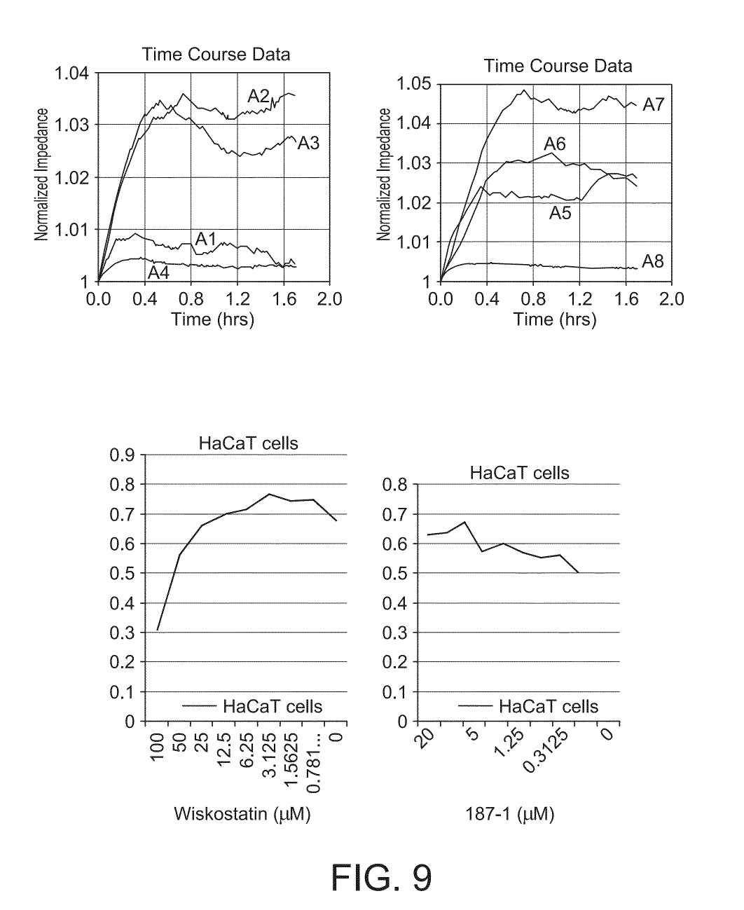

Finally, all the effects of Wiskostatin and 187-1 on the aforementioned cell functions were achieved at concentrations that are far below those that may cause toxicity and growth changes (FIG. 9C bottom).

Knocking Down n-WASP from Keratinocytes Drastically Increased the Migration of the Cells

Based on the secondary structure of nWASP mRNA (FIG. 2), three sites were found suitable for targeting. Three anti-nWASP transgenes were created. In transfecting the HaCaT cells, transgenes-2 and -3 were highly active (FIG. 2) and the cells that carried these two transgenes were subsequently used for testing.

As shown in FIG. 9 (top left), cells that carried anti-nWASP transgenes and so lost nWASP mRNA had dramatically increased rate of healing/migration when compared with the control cell. Using HGF as a migration inducing agent, it was found that HGF increased the migration of control HaCaT cells and also has some limited effect on the nWASP transgene carrying cells (FIG. 9 top right)
Impact of nWASP Over-Expression on In Vitro Angiogenesis and Migration of Vascular Endothelial Cells

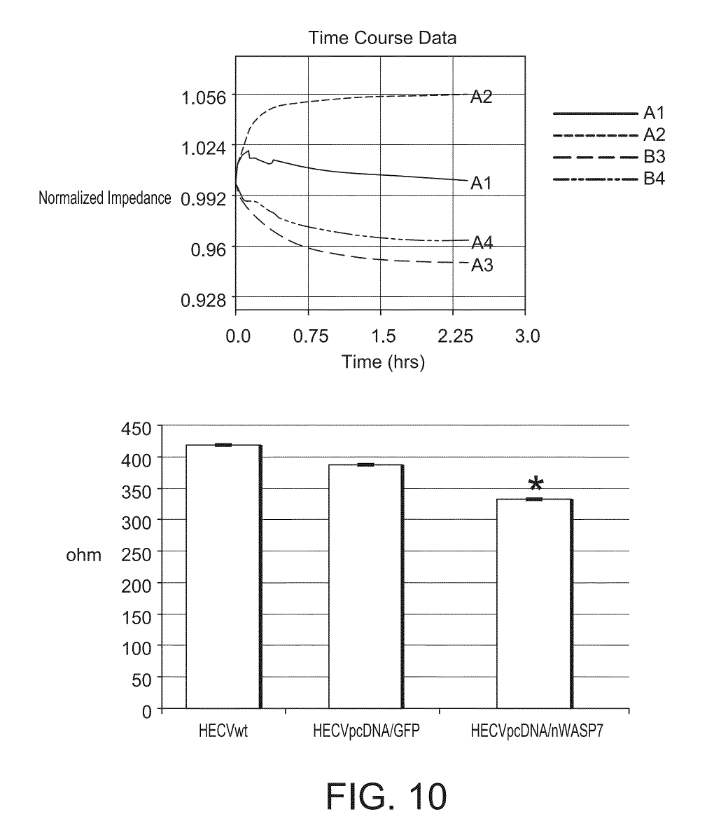

When HECV cells were forced to express nWASP, there was a significant reduction in the migration as shown in FIGS. 10A and 10b.

Using in vitro tubule formation assays, it was found that neither 187-1 nor Wiskostatin had a major impact on the tubule formation from HECV cells, a cell line that is negative for nWASP expression. However, as shown in FIG. 11A, when HECV cells were transfected and forced to express nWASP, their responses to the inhibitors changed substantially, particularly with 187-1, in that the presence of 187-1 markedly increased the formation of microtubules in nWASP expression HECV cells. FIG. 11B shows tubule forming after 48 hours. Both nWASP and 187-1 now show a strong stimulatory effect on tubule forming, in HECV cells that were forced to express nWASP.
nWASP Inhibitors and Expansion of Chronic Wound Tissues

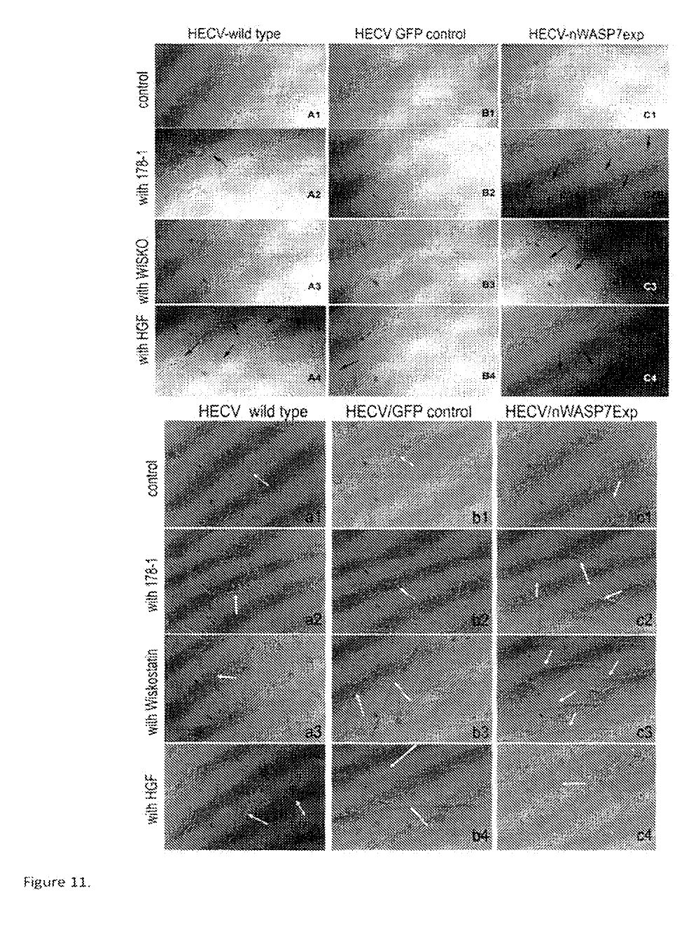

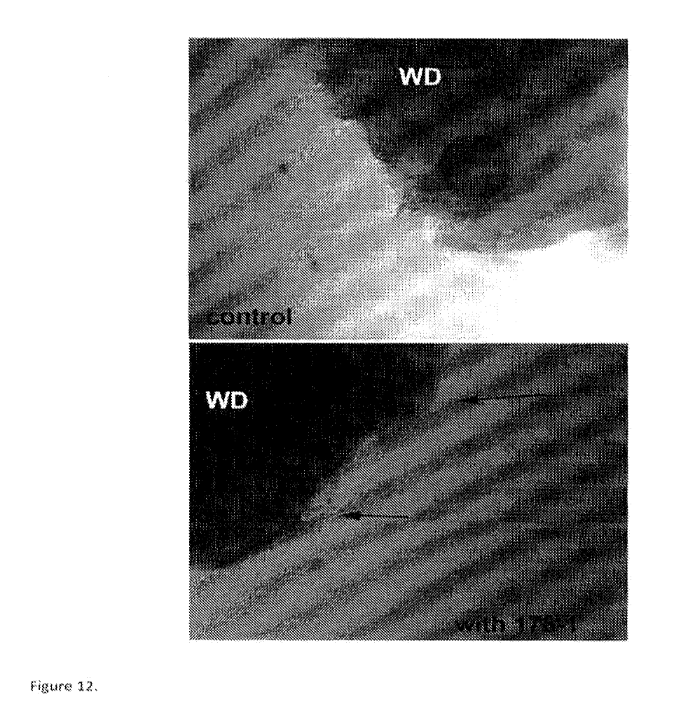

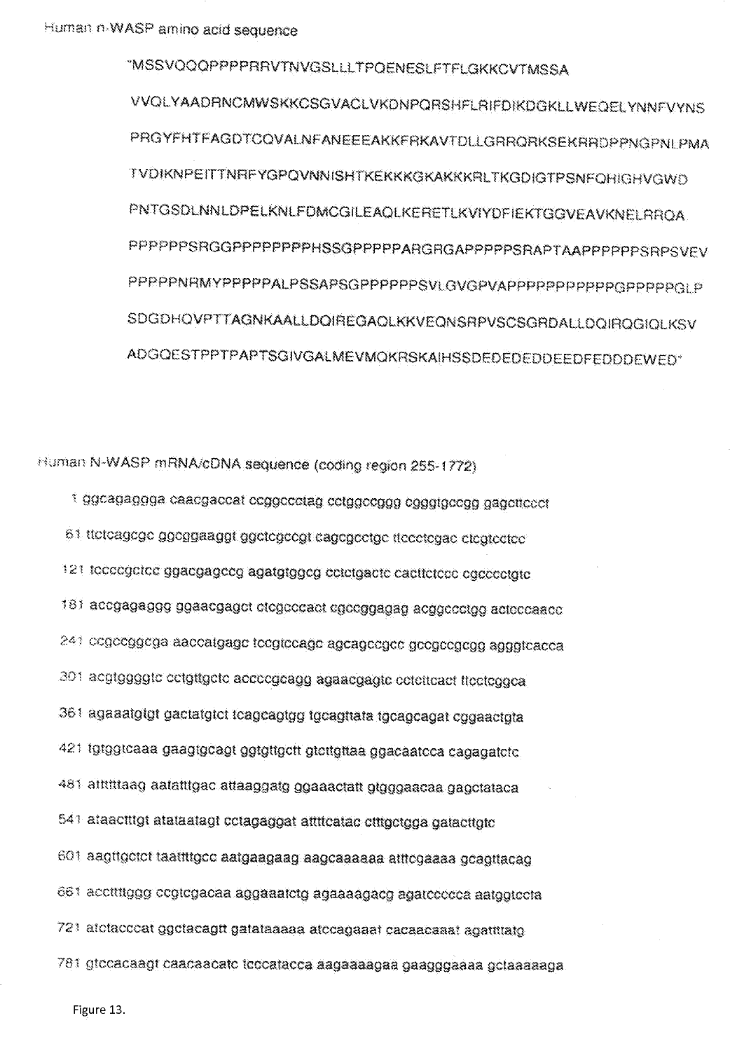

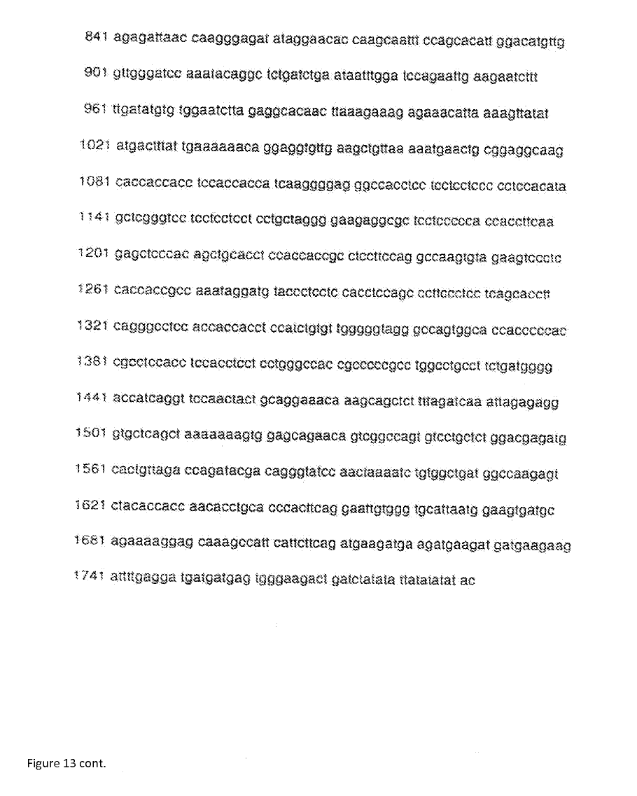

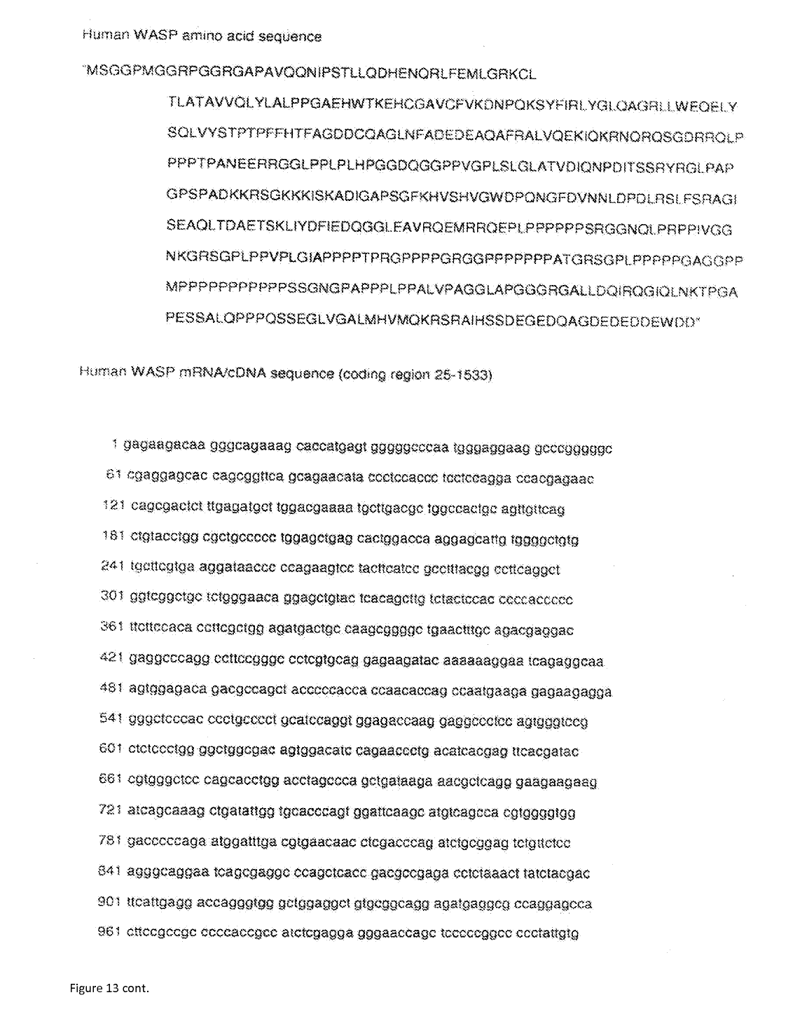

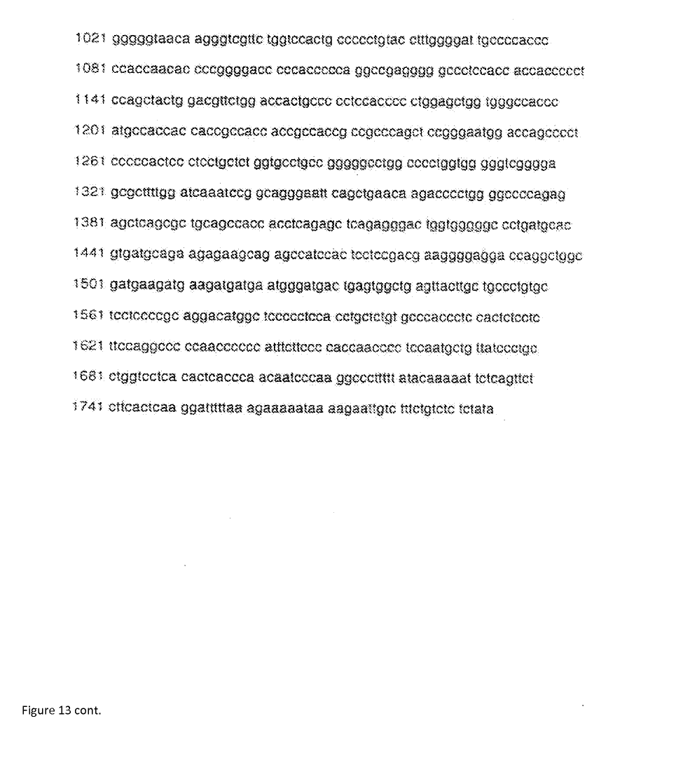

We tested the impact of nWASP inhibitors on the expansion of wound tissues using an ex vivo model that we established. As shown in FIG. 12, inclusion of 187-1 in the 3-D system resulted in expansion of the chronic tissues, whereas no expansion was seen in control conditions over the same period.

Validation Study on Chronic Wound Tissues Revealed a Significant Over-Expression of nWASP in Chronic Non-Healing Wound Tissues

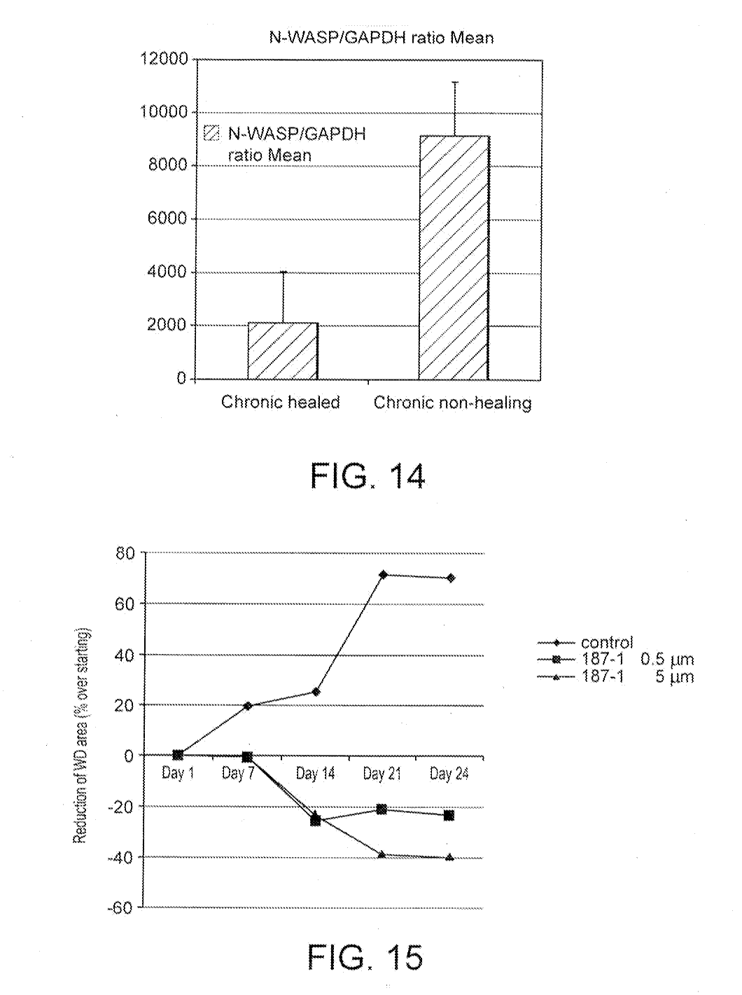

Using an independent cohort of chronic tissues, we have shown wounds that failed to heal had a significantly higher levels of nWASP that which had healed (p<0.05) (FIG. 14).

### 187-1 and Wiskostatin are Well Tolerated

We have delivered the compounds systemically on a daily basis in our tolerant test, for a two week period in athymic CD-1. Throughout the study, we did not observe any side effects. There was no weight loss and no signs of any changes in the skin in any of the groups. The compounds were also well tolerated in the db/db strain, in that administration on alternative date rendered no side effects.

187-1, Administered Systemically, Accelerated Wound Healing without Producing any Side Effects

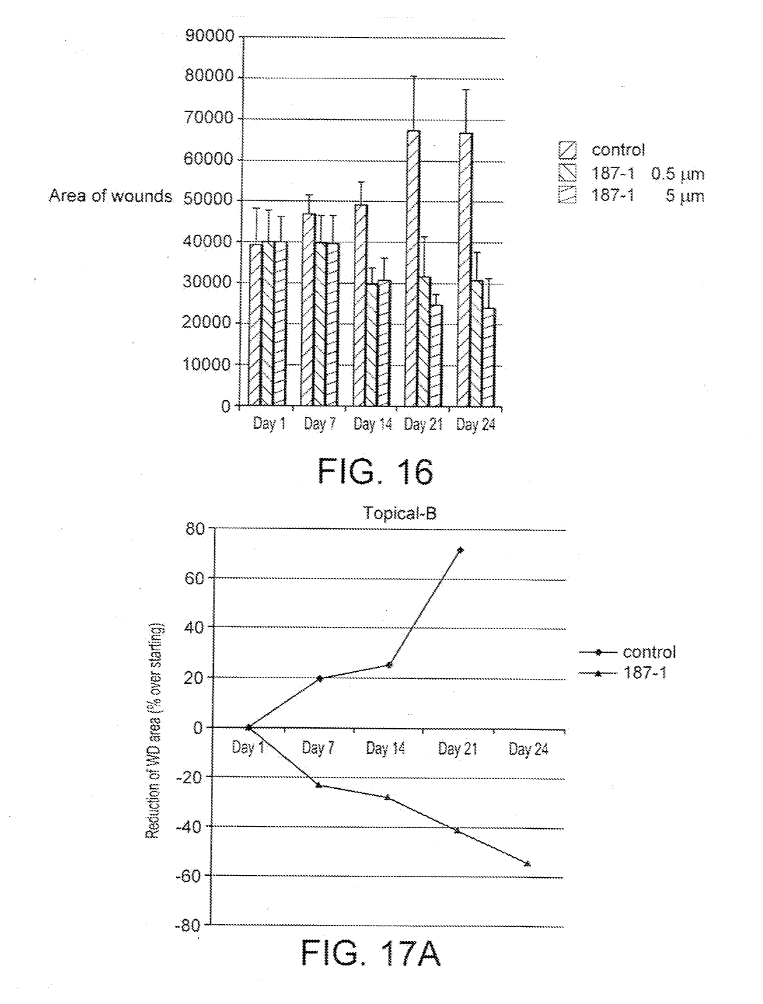

Two concentrations of 187-1 were given systemically, 0.5 and 5 μM (equivalent to 0.89 g/kg/day and 8.9 g/kg/day). After two weeks, wounds in the treated group were significantly smaller than the control group as shown in FIG. 15 (p=0.037 and p=0.04, control vs 187-1, 0.5 and 5 μM respectively, 2-Way ANOVA with Bonferroni model). FIG. 16 showed the change of wound area.

187-1, Administered Topically, Accelerated Wound Healing without Producing any Side Effects

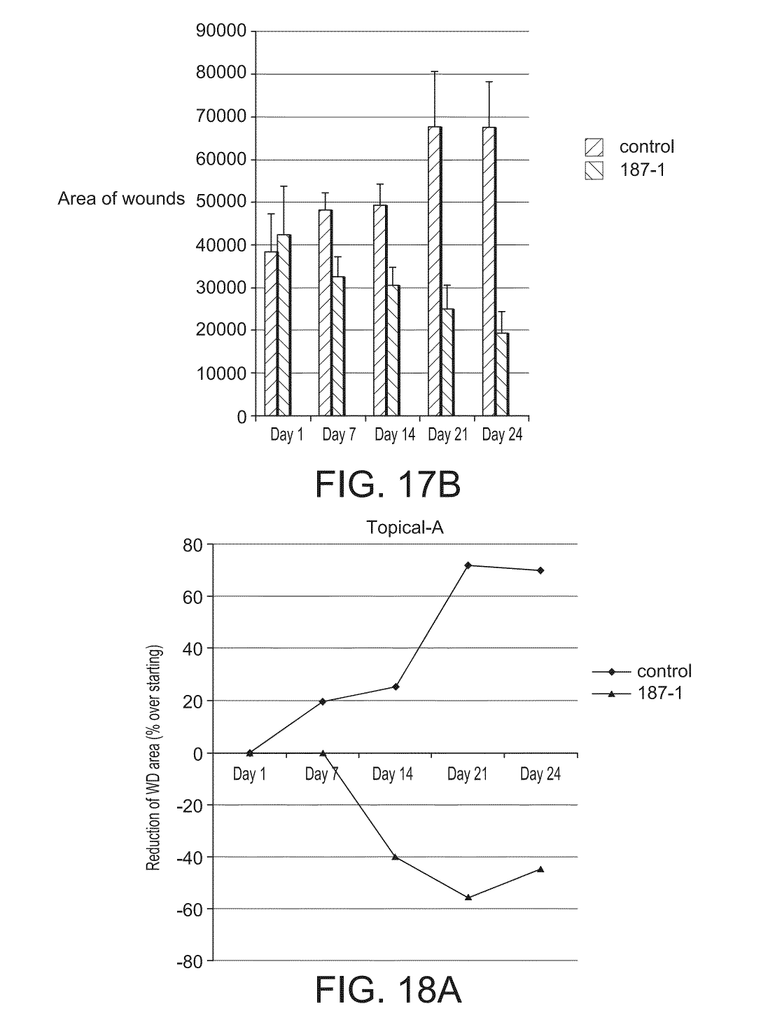

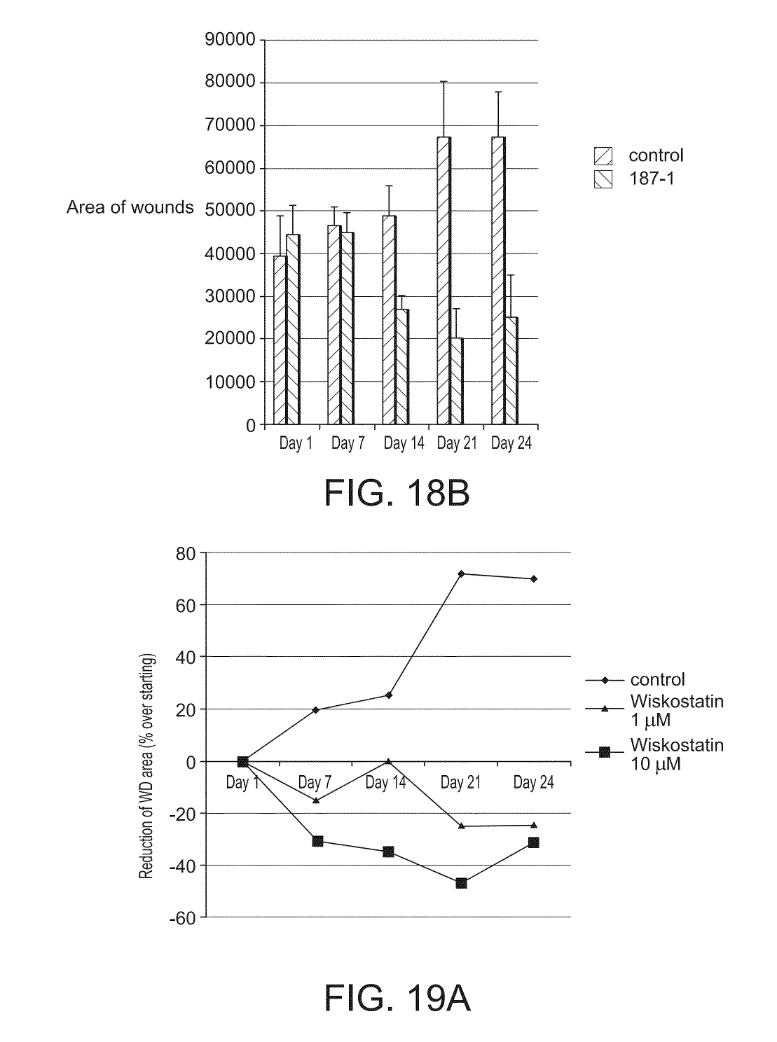

Topical application of 187-1 in both gels A and B showed a significant effect after three weeks, (p=0.028 and p=0.045, control vs 187-1, in topical-B and Topical-A applications respectively, 2-Way ANOVA with Bonferroni model) (FIGS. 17 and 17b, 18 and 18b).

Wiskostatin, Systemically Delivered, Significantly Accelerated Wound Healing without Producing any Side Effects

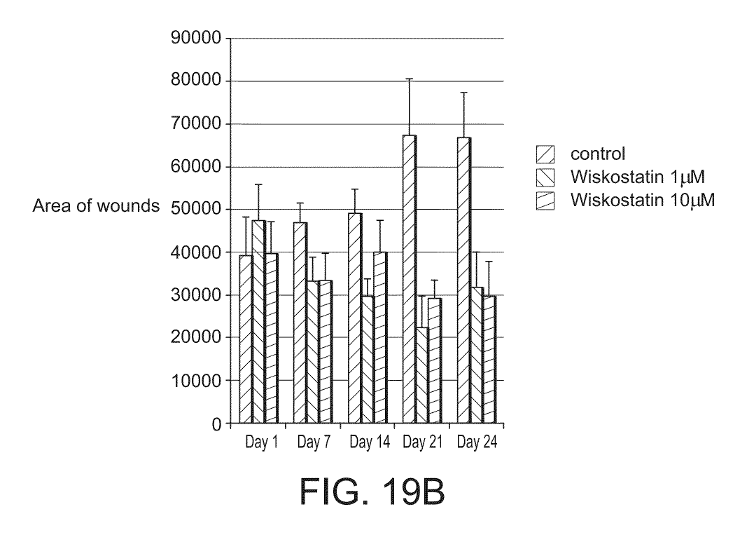

At 1 and 10 μM, Wiskostatin had significantly reduced the size of the wounds in comparison with control (p=0.017 and p=0.04, control vs Wiskostatin, 1 and 10 μM respectively, 2-Way ANOVA with Bonferroni model) after three weeks (FIGS. 19 and 19b).

Topical Application of Wiskostatin Also Showed a Significant Effect after Three Weeks

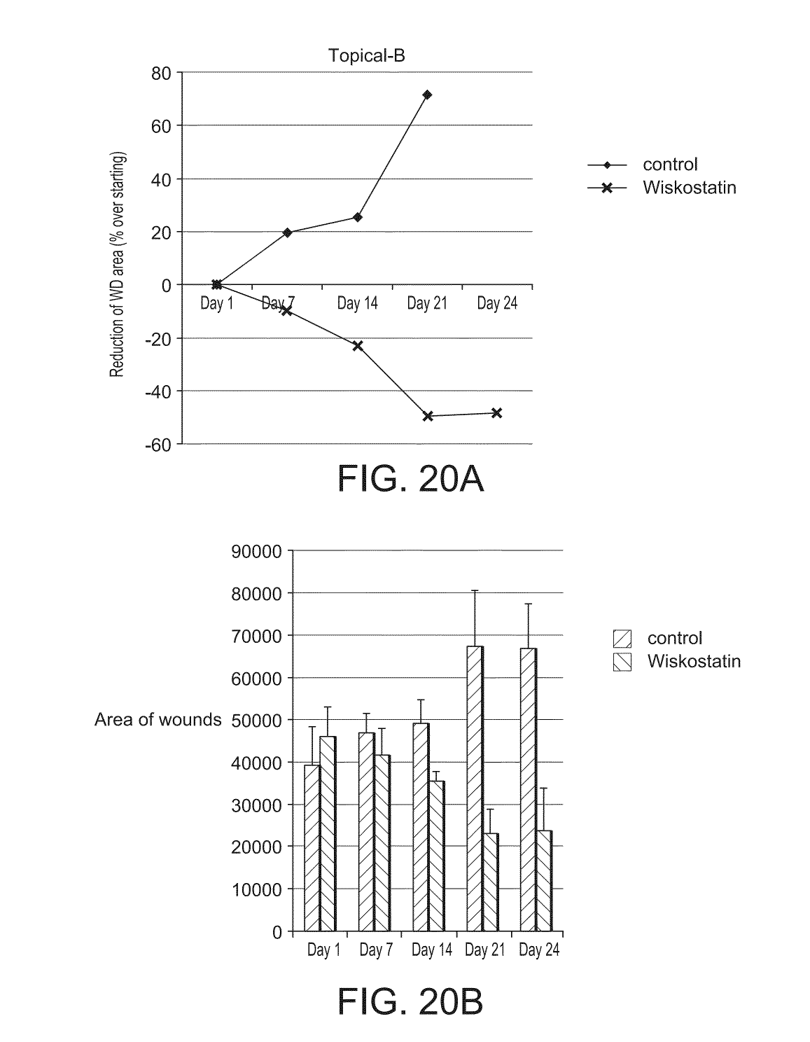

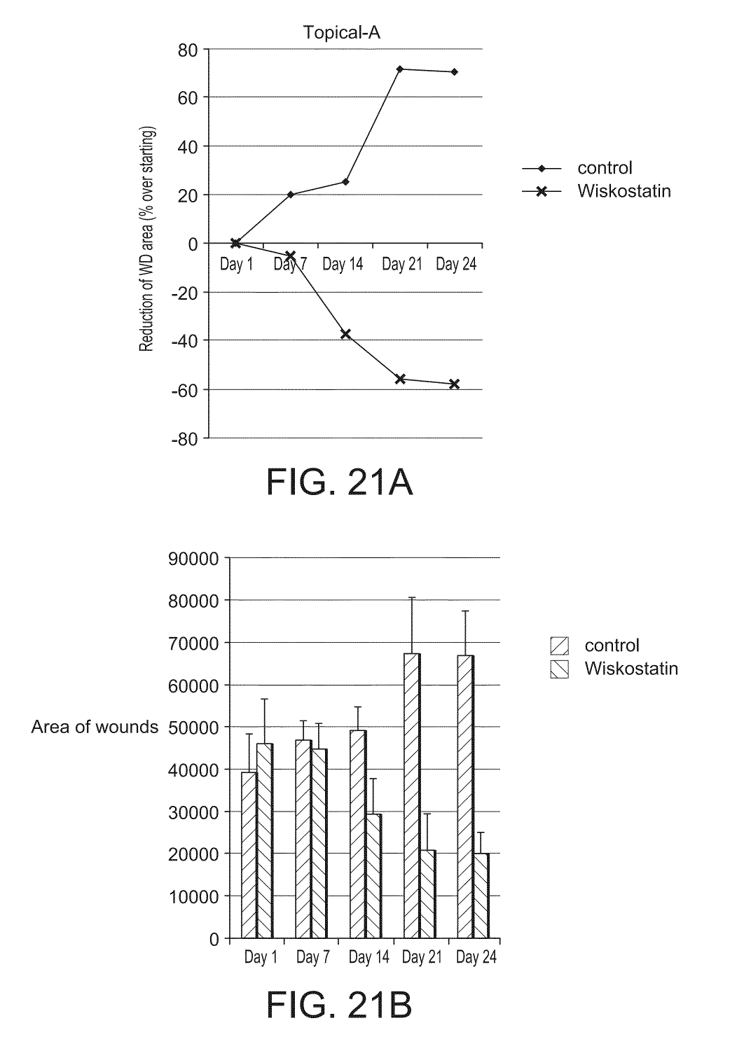

Using either carrier gel Wiskostatin showed a significant effect (p=0.044 and p=0.044, control vs 187-1, in topical-B and Topical-A applications respectively, 2-Way ANOVA with Bonferroni model) (FIGS. 20 and 20b, 21 and 21b).

### Summary

The main findings of the present study can be summarised in the following:

Abnormal/chronic human wounds have increased levels of nWASP as demonstrated by quantitative transcript analysis; and using an exogenous nWASP inhibitor, such as Wiskostatin, can increase the migration and healing speed of wounded tissue or using knock down nWASP transcripts can also increase the migration and healing speed of wounded tissue.

These findings collectively show that nWASP is critical in controlling the migration and healing of wounds. Together with the increased expression of nWASP in abnormal/chronic wounds, it is plausible to suggest that high levels of nWASP in a given wound may indicate that nWASP hampers the healing process of the wound, which may primarily be due to reduced ability of cells type, to migrate into the wound. Thus, both in vitro and clinical data point to nWASP being an important therapeutic target in abnormal/chronic wounds.

What is claimed:

1. A method for treating a mammalian wound, comprising administering to the wound a topical preparation comprising:
an inhibitor of at least one of a neural Wiskott Aldrich Syndrome Protein (nWASP) gene expression and an nWASP protein activity, or an inhibitor of activity of a protein that is at least 75% homologous to the nWASP protein and that decreases actin assembly; and
a pharmaceutically or veterinarily acceptable carrier.

2. The method of claim 1, including administering a topical preparation comprising an inhibitor of activity of an nWASP protein comprising SEQ ID NO: 13.

3. The method of claim 1, including administering a topical preparation comprising an inhibitor of expression of an nWASP gene comprising SEQ ID NO: 14.

4. The method of claim 1, including administering a topical preparation comprising an inhibitor of activity of a protein that is at least 90% homologous to the nWASP protein and that decreases actin assembly.

5. The method of claim 1, wherein said inhibitor is selected from the group consisting of: a nWASP binding agent that binds, either reversibly or irreversibly, to inhibit protein function such as an antibody or a known, or synthesized, nWASP antagonist; or an agent that works upstream or downstream of the nWASP signalling mechanism to inhibit nWASP function.

6. The method of claim 1, including administering a topical preparation comprising an inhibitor selected from one of Wiskostatin or 187-1.

7. The method of claim 1, including administering a topical preparation comprising an nWASP gene expression inhibitor selected from the group consisting of anti-sense DNA or RNA, siRNA, and ribozymes;
further wherein said inhibitor is provided naked or in the form of a plasmid or a viral vector.

8. The method of claim 7, including administering a topical preparation comprising an inhibitor that is an anti-nWASP ribozyme/RNA transgene selected from the group consisting of:
transgene 1
(SEQ ID NO: 17)
5′Ctgcaggagttctttgaccacatacagttccctgatgagtccgtgagg
acgaaatctgctgcatataactgcaccacactagt′3;
transgene 2
(SEQ ID NO: 18)
5′Ctgcagacaagcaacaccactgcacttctttctgatgagtccgtgagg
acgaaaccacatacagttccgatctgctgcatataactagt′3;
and
transgene 3
(SEQ ID NO: 19)
5′Ctgcaggtgcagctgtgggagctcttctgatgagtccgtgaggacgaaa
aggtggtgggggaggagcgcctcttcccctagcctagt′3.

9. The method of claim 1, including administering the topical preparation to a chronic wound.

10. The method of claim 1, including administering the topical preparation to a human chronic wound.

11. The method of claim 1, including administering the topical preparation in a formulation suitable for application to a dressing to be applied to the chronic wound or impregnation in a dressing to be applied to the chronic wound.

12. A method for treating a mammalian wound, comprising administering to the wound a topical preparation comprising:
an inhibitor of an nWASP protein activity, or an inhibitor of activity of a protein that is at least 75% homologous to the nWASP protein and that decreases actin assembly; and
a pharmaceutically or veterinarily acceptable carrier.

13. The method of claim 12, including administering a topical preparation comprising an inhibitor of activity of an nWASP protein comprising SEQ ID NO: 13.

14. The method of claim 12, including administering a topical preparation comprising an inhibitor of activity of a protein that is at least 90% homologous to the nWASP protein and that decreases actin assembly.

15. The method of claim 12, wherein said inhibitor is selected from the group consisting of: a nWASP binding agent that binds, either reversibly or irreversibly, to inhibit protein function such as an antibody or a known, or synthesized, nWASP antagonist; or an agent that works upstream or downstream of the nWASP signalling mechanism to inhibit nWASP function.

16. The method of claim 12, including administering a topical preparation comprising an inhibitor selected from one of Wiskostatin or 187-1.

17. The method of claim 12, including administering the topical preparation to a chronic wound.

18. The method of claim 12, including administering the topical preparation to a human chronic wound.

19. The method of claim 12, including administering the topical preparation in a formulation suitable for application to a dressing to be applied to the chronic wound or impregnation in a dressing to be applied to the chronic wound.

20. A method for treating a mammalian wound, comprising administering to the wound a topical preparation comprising one of Wiskostatin or 187-1 and a pharmaceutically or veterinarily acceptable carrier.

21. The method of claim 20, including administering the topical preparation to a chronic wound.

22. The method of claim 20, including administering the topical preparation to a human chronic wound.

23. The method of claim 20, including administering the topical preparation in a formulation suitable for application to a dressing to be applied to the chronic wound or impregnation in a dressing to be applied to the chronic wound.
